(19) United States
(12) Patent Application Publication
(10) Pub. No.: US 2015/0071880 A1
(43) Pub. Date: Mar. 12, 2015

(54) TREATMENT OF EPITHELIAL LAYER LESIONS

(71) Applicant: Minas Theodore Coroneo, Vaucluse (AU)

(72) Inventor: Minas Theodore Coroneo, Vaucluse (AU)

(21) Appl. No.: 14/546,363

(22) Filed: Nov. 18, 2014

Related U.S. Application Data
(63) Continuation of application No. 11/364,874, filed on Feb. 27, 2006.
(60) Provisional application No. 60/656,164, filed on Feb. 25, 2005.

(51) Int. Cl. A61K 38/21; A61K 31/203

(52) U.S. Cl. CPC A61K 38/212; A61K 31/203
USPC 424/85.7

(57) ABSTRACT

Methods for the treatment of lesions originating in an epithelial layer, particularly diseases of the epithelium of the skin, bladder, oral mucosa, vagina and cervix are described. Also described are uses and formulations for the treatment of lesions originating in an epithelial layer.

### CROSS-REFERENCES TO RELATED APPLICATIONS

This application is a continuation of co-pending U.S. application Ser. No. 11/364,874, filed Feb. 27, 2006, which application claims the benefit under 35 U.S.C. §119(e) of U.S. Provisional Patent Application No. 60/656,164, filed Feb. 25, 2005. The disclosures of these applications are incorporated herein by reference in their entireties.

### TECHNICAL FIELD

The present invention relates to the treatment of lesions in the epithelial layer of the body.

Particularly, the invention relates to compositions, uses and non-surgical therapeutic treatments for lesions originating in the epithelial layer of the body with a combination of interferon alpha (α) and retinoids.

### BACKGROUND OF THE INVENTION

Epithelial tissue comprises the cellular lining that covers the external and internal body surfaces, and is classified according to the number of layers making up the lining, as well as the shape of the cells within the lining (Dorland's Pocket Medical Dictionary 24^{th} Ed., W.B. Saunders Company, Philadelphia). Epithelial tissues come in three basic types: squamous, cuboidal and columnar. These three types of tissue are seen in either simple (only one cell layer thick) or stratified (many cells in thickness) arrangements. Cell types other than epithelial cells are also found within the epithelial layer; for example, the epithelial layer of the skin contains melanocytes at the border of the epidermis and the dermis. Melanocytes are of neural crest origin, unlike epithelial cells, which originate from the ectodermal ridge.

Proliferative lesions originating in the epithelial layer can occur in many tissues of the body, and are generally characterised by cells within the epithelial layer dividing in an uncontrolled manner. When this cell division reaches a particular rate, the lesion may be classified as a tumour, and in particular, a carcinoma, the latter being a term relating to a malignant tumour that originates in the epithelium.

A key example of lesions of the epithelial layer are pre-cancerous and cancerous lesions of the skin. There are a variety of types of skin cancers, including basal cell carcinomas, which are a relatively slow-growing type of cancer; squamous cell carcinoma, which is the second-most common type of skin cancer; and melanoma, which is the least common but most serious type of skin cancer. This latter type of skin cancer involves proliferation of melanocytes within the epithelial layer of the skin. The primary treatment for all skin cancers or pre-cancerous lesions of the skin is surgery, which may be accompanied by chemotherapy or radiation where this is a high risk of recurrence of the skin cancer.

Carcinoma of the bladder primarily originates in the bladder lining, which consists of a mucous layer of surface cells that expand and deflate (transitional epithelial cells), smooth muscle, and a fibrous layer. Tumors are categorized as low-stage (superficial) or high-stage (muscle invasive). Treatment for bladder cancer depends on the stage of the disease, the type of cancer, and the patient's age and overall health. Treatment options include surgery, chemotherapy, radiation, and immunotherapy. In some cases, treatments are combined (eg. surgery or radiation and chemotherapy, preoperative radiation).

Cervical dysplasia or neoplasia is characterised by abnormal growth of the epithelial layer of the cervix which is generally categorised according to three stages. The usual treatment for this type of cancer is colposcopy and cryosurgery.

Hyperproliferative lesions of the vaginal epithelial layer or vaginal neoplasias are divided into five stages, with stage 0 representing the earliest, pre-cancerous stage, where there may be cancerous cells but these are confined to a particular site. Stages 2 and beyond involve growth of the cancer outside the vaginal tissue. Again, treatment of vaginal neoplasias involve surgery in combination with chemotherapy and/or radiation therapy.

Leukoplakia is an oral lesion of the epithelial layer of the mouth that appears as a whitish patch that cannot be characterised clinically or pathologically as any other disease, and is not associated with any physical or chemical causative agent, other than tobacco use. It usually occurs on the mucous epithelial membranes of the cheeks, gums or tongue, and may develop fissures. It is also known as idiopathic leukoplakia, and is associated with a high risk of developing into cancer. The treatment of cancerous leukoplakia generally involves surgical removal of the growth using a scalpel, laser or cryoprobe.

Thus, notwithstanding surgical removal of lesions originating in the epithelial layer of the body, with their attendant costs and risk of complications, many of these cancer types regrow. The treatments to prevent regrowth of cancerous lesions originating in the epithelial layer are associated with side effects, and do not prevent effectively regrowth in many cases.

Retinoids have been used to reduce the cohesiveness of hyper-proliferative keratinocytes in leukoplakias, and are thought to reduce the possibility for malignant degeneration of the lesions, as they modulate keratinocyte differentiation. The routes for delivering these drugs have included topical and systemic administrations (Lodi et al., Cochrane Review, in The Cochrane Library, Issue 3, 2004, Chichester UK: John Wiley & Sons). While the treatment of oral leukoplakias with retinoids has reportedly assisted in treating the lesion, recurrence rates remain high (Lodi et al., above).

### SUMMARY OF THE INVENTION

Unexpectedly, it has been found by the present inventor that the combination of interferon α and retinoid is effective in the treatment of lesions originating in the epithelial layer of the body, particularly diseases of the epithelium selected from pre-cancerous and cancerous lesions of the skin, bladder, vaginal and cervical neoplasias and oral leukoplakia, in a patient in need of such treatment.

The combined treatment of lesions originating in the epithelial layer with interferon α and retinoid may avoid the need for surgery for lesion removal and is effective in preventing lesion recurrence.

In accordance with a first aspect of the present invention there is provided a method for the treatment of lesions originating in the epithelial layer of the body, particularly diseases of the epithelial layer selected from pre-cancerous and cancerous lesions of the skin, bladder, vaginal and cervical neoplasias and oral leukoplakia in a patient in need of such treatment, which comprises administering to the lesion of the patient a therapeutically effective amount of interferon α and retinoid.

In accordance with a second aspect of the present invention there is provided a formulation of interferon α and retinoid for the combination treatment of lesions originating in the epithelial layer, particularly diseases of the epithelium of the skin, bladder, oral mucosa, vagina and cervix.

In accordance with a third aspect of the present invention there is provided use of interferon α and retinoid in the manufacture of medicaments for the combination treatment of lesions originating in the epithelial layer, particularly diseases of the epithelium of the skin, bladder, oral mucosa, vagina and cervix.

Preferably, the formulations and medicaments of the invention are for topical administration to the lesion originating in the epithelial layer.

### DETAILED DESCRIPTION

As used herein, the term “retinoid” refers to a natural or synthetic vitamin A analogue or other compound is an agonist of one or both nuclear retinoic acid receptor (RAR) and retinoic X receptor (RXR) each of which is encoded by three separate genes designated α, β, and γ (Peck, G. L., and Di Giovanni, J. J. “The retinoids: biology, chemistry and medicine”, pp 631-658, Raven Press, New York, (1994)) the contents of which are incorporated herein by reference.

Preferred retinoid includes retinoic acid, all-trans retinoic acid, 9-cis-retinoic acid and

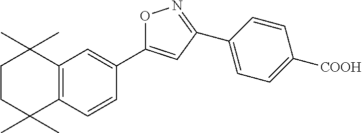 [SMILES: CC1(C)CCC(C)(C)c2cc(-c3cc(-c4ccc(C(=O)O)cc4)no3)ccc21]

It has surprisingly been found by the present inventor that the combination of interferon α and retinoid is effective in the treatment of lesions originating in the epithelial layer, particularly diseases of the epithelium selected from pre-cancerous and cancerous lesions of the skin, bladder, vaginal and cervical neoplasias and oral leukoplakia, in a patient in need of such treatment.

The present invention in its various aspects includes the non-surgical treatment of the aforementioned lesions originating in the epithelial layer, in particular amelioration of the lesion without recurrence, in contrast to the prior art.

The term “lesion originating in the epithelial layer” as used herein refers to lesions associated with hyperproliferative cellular division, and encompasses both non-cancerous and cancerous lesion affecting any type of epithelial cell lining internal and external body surfaces, and include, but are not limited to, ciliated epithelia, columnar epithelia, cuboidal epithelia, germinal epithelia, glandular epithelia, laminated epithelia, olefactory epithelia, pseudostratified epithelia, seminiferous epithelia, simple epithelia, squamous epithelia, stratified epithelia and transitional epithelia.

In accordance with a first aspect of the present invention there is provided a method for the treatment of lesions originating in the epithelial layer, particularly diseases of the epithelium selected from pre-cancerous and cancerous lesions of the skin, bladder, vaginal and cervical neoplasias and oral leukoplakia, in a patient in need of such treatment, which comprises administering to the lesion of the patient a therapeutically effective amount of interferon α and retinoid.

By “effective amount,” in the context of treating or preventing a condition is meant the administration of that amount of active to an individual in need of such treatment or prophylaxis, either in a single dose or as part of a series, that is effective for treatment of, or prophylaxis against, that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

The retinoids and interferons used in the compositions and methods of the invention include all salts, such as acid addition salts, anionic salts and zwitterionic salts, and in particular include pharmaceutically acceptable salts as would be known to those skilled in the art. The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, tosylate, citrate, bicarbonate and carbonate.

Pharmaceutically acceptable salts include those formed from: acetic, ascorbic, aspartic, benzoic, benzenesulphonic, citric, cinnamic, ethanesulphonic, fumaric, glutamic, glutaric, gluconic, hydrochloric, hydrobromic, lactic, maleic, malic, methanesulphonic, naphthoic, hydroxynaphthoic, naphthalenesulphonic, naphthalenedisulphonic, naphthaleneacrylic, oleic, oxalic, oxaloacetic, phosphoric, pyruvic, p-toluenesulphonic, tartaric, trifluoroacetic, triphenylacetic, tricarballylic, salicylic, sulphuric, sulphamic, sulphanilic and succinic acid.

The term “pharmaceutically acceptable derivative” refers to a derivative of the active compound that upon administration to the recipient is capable of providing directly or indirectly, the parent compound or metabolite, or that exhibits activity itself and includes for example phosphate derivatives and sulphonate derivatives. Thus, derivatives include solvates, pharmaceutically active esters, prodrugs or the like. This also includes derivatives with physiologically cleavable leaving groups that can be cleaved in vivo to provide the compounds of the invention or their active moiety. The leaving groups may include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted compounds of the invention are readily cleavable to the corresponding hydroxy substituted compounds.

The interferon α and retinoid may be administered by topical administration over the lesion for a treatment period until the particular condition being treated resolves. The interferon α and retinoid may be administered sequentially or concurrently. Preferably, the interferon α is administered one or more times daily, and the retinoid is administered sequentially on every second day during treatment. The time interval between interferon α administration and retinoid administration in an embodiment of this invention is up to about 36 hours, such as 24 to 36 hours.

The interferon α is preferably interferon α, more preferably α-2b, such as that produced by Schering. The interferon α may be administered onto the lesion in an amount from about 1×10⁴ to about 1×10⁶ international units (IU) per day, more preferably 1×10⁵ to 1×10⁶ IU per day, still more preferably 1×10⁶ IU per day.

Retinoid may be administered to the lesion in a topical composition. Retinoid may be administered onto the lesion in an amount of from about 0.05 μg to about 50 μg every second day, for example 1-20 μg, 5-15 μg or 5-10 μg. Retinoid topical compositions may contain, for example a dosage of 0.005 to 0.15% retinoid in a pharmaceutically compatible vehicle. A particularly preferred dosage form is from 1 to 2 drops every second day of an 0.01% w/w retinoid topical epithelial-layer lesion composition.

Epithelial composition vehicles comprise pharmaceutically acceptable carriers and excipients. By “pharmaceutically-acceptable carrier” is meant a solid or liquid filler, diluent or encapsulating substance which may be safely used for administration of the medicament or pharmaceutical preparation to a patient. Depending upon the particular route of administration, a variety of pharmaceutically-acceptable carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Controlled- or sustained-release formulations of a pharmaceutical composition of the invention may be made using conventional technology. For example, they can also be provided in the form of polymeric or lipid vesicles or nanospheres or microspheres or of polymeric patches and of hydrogels which make possible controlled release of the active agents.

A formulation of a pharmaceutical composition of the invention suitable for topical administration to an epithelial layer may be prepared, packaged, or sold in the form of a discrete solid dose unit including, but not limited to, a tincture, an ointment, a cream, a salve, a powder, an impregnated pad, a gel, a spray, a lotion, a suspension, a tablet, a hard or soft capsule, a cachet, a troche, or a lozenge, each containing a predetermined amount of the active ingredients. Other formulations suitable for topical administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the invention which are suitable for topical administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

Where interferon α and retinoid are administered concurrently, they may be included in the same topical composition for administration onto the lesion originating in the epithelial layer.

Prior to the treatment of lesions originating in the epithelial layer in a patient by administration to the lesion of a combination of interferon α and retinoid, or during such treatment, interferon α may be administered by intralesion injection particularly during an initial treatment period, in an amount from 0.5 to 5 million international units, preferably from 1 to 3 million international units administered interlesionally for seven to 14 days. For example, interlesional interferon α may be administered twice weekly over a two week period from one to three times per week, preferably twice per week over a two week period.

The retinoid is an agonist of one or both of the retinoic acid receptor (RAR) and retinoid X receptor (RXR). Standard in vitro tests for receptor binding may be carried out to determine binding to RAR and RXR, for example as described in De Simoni et al, Pure Appin. Chem. Vol 73, No. 9, pp 1437-1444, 2001. RAR and RXR retinoid agonists, including both RAR specific and RXR specific agonists have been previously identified. See for example, WO 94/15902, WO 93/21146, WO 94/15901, WO 94/12880, WO 94/17796, WO 94/20093, WO 96/05165 and International Application No. PCT/US93/10166; European Patent Applications Nos. 87110303.2, 87309681.2 and EP 0718285; U.S. Pat. Nos. 4,193,931, 4,539,134, 4,801,733, 4,831,052, 4,833,240, 4,874,747, 4,879,284, 4,898,864, 4,925,979, 5,004,730, 5,124,473, 5,198,567, 5,391,569, Reissue Pat. No. 33,533, U.S. Pat. Nos. 5,693,493, 5,968,493, 6,030,964, 6,133,309, 6,147,244, 6,593,493; H. Kagechiki et at “Retinobenzoic Acids. 2. Structure-Activity Relationship of Chalcone-4-carboxylic Acids and Flavone-4′-carboxylic Acids”, J. Med. Chem., 32:834 (1989); H. Kagechika et at “Retinobenzoic Acids. 3. Structure-Activity Relationships of Retinoidal Azobenzene-4-carboxylic Acids and Stilbene-4-carboxylic Acids”, J. Med. Chem., 32:1098 (1989); H. Kagechika et at “Retinobenzoic Acids. 4.4 Conformation of Aromatic Amides with Retinoidal Activity. Importance of trans-Amide Structure for the Activity”, J. Med. Chem. 32:2292 (1989); M. Boehm et al, J. Med. Chem. 37:2930 (1994); M Boehm et al, J. Med. Chem. 38:3146 (1995); E. Allegretto et al, Journal of Biol. Chem., 270:23906 (1995); R. Bissonnette et al, Mol. & Cellular Bio., 15:5576 (1995); R. Beard et al, J. Med. Chem., 38:2820 (1995); M. I. Dawson et at “Effect of Structural Modifications in the C7-C11 Region of the Retinoid Skeleton on Biological Activity in a Series of Aromatic Retinoids”, J. Med. Chem., 32:1504 (1989); and Simoni et al, Pure Appin. Chem., Vol 73, No. 9, pp 1437-1444, 2001. The RAR and RXR retinoid agonists referred to in the abovementioned patent applications, patents and scientific articles may be used in the present invention and have direct application as retinoids in the various therapeutic aspects of this invention described herein. Methods for preparation of retinoids, dosage ranges and compositions/formulations are described in the various patent applications/patents and scientific articles referred to above, and are incorporated herein by reference.

Preferred retinoids include retinoic acid, all-trans retinoic acid, 9-cis-retinoic acid and

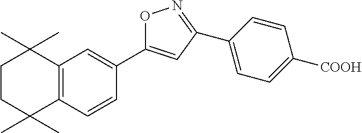 [SMILES: CC1(C)CCC(C)(C)c2cc(-c3cc(-c4ccc(C(=O)O)cc4)no3)ccc21]

Compositions may be prepared in standard fashion by formulating interferon α, particularly interferon α-2b, in association with one or more pharmaceutically acceptable carriers and excipients. Similarly, the retinoid may be formulated into a composition with one or more pharmaceutically acceptable carriers and excipients. Where a combination composition is prepared, both the interferon α and the retinoid are formulated together with one or more pharmaceutically acceptable carriers and excipients.

In accordance with a second aspect of the present invention there is provided a formulation of interferon α and retinoid for the combination treatment of lesions originating in the epithelial layer, particularly lesions of the epithelium of the skin, bladder, oral mucosa, vagina and cervix.

Formulations include one or more pharmaceutically acceptable carriers and excipients as are known in the art. Formulations of interferon α and retinoid may be prepared separately for administration. Alternatively, combined formulations of both interferon α and retinoid may be prepared.

Treatment duration according to the various aspects of the present invention are carried out until the disease condition has resolved, or in the alternative is carried out for a period of from one to six months after which treatment would be stopped. If necessary, the lesion may be surgically removed. However, should the lesion completely resolve by this time, which is desirable, surgery is thus not required. Furthermore, if the lesion reappears, treatment can be restarted. Where lesions are neoplastic, and in particular where they are of large size such that they cause discomfort, they are generally surgically removed at the end of treatment. The treatment of the present invention may cause extensive necrosis of the lesion thus facilitating surgical removal. Moreover the necrosis of the lesion is believed to prevent lesion recurrence. Treatment according to the present invention is generally in the range from one to six months, although shorter or longer treatment durations may be required depending on the severity of the lesion.

In accordance with a third aspect of the present invention there is provided use of retinoid and interferon α in the manufacture of medicaments for the treatment of lesions originating in the epithelial layer, particularly diseases of the epithelium of the skin, bladder, oral mucosa, vagina and cervix.

Without wishing to be bound by theory, the inventor believes the combined use of interferon α and retinoid for lesions originating in the epithelial layer, which is effective in the treatment of lesions originating in the epithelial layer, whereas individual use of such agents is not effective, may be due to a combination of inducement of differentiation of lesion tissue, apoptosis of lesion tissue and inhibition of blood vessels/fibrous tissue associated with lesions.

The invention will now be described with reference to the following non-limiting examples.

### EXAMPLES

### Example 1

### Preparation of Retinoic Acid Formulation for Topical Application

| | |
|---|---|
| Retinoic Acid 0.01% eye drops 10 mL | Source of formula: |
| In glass amber eye dropper bottles | Martindale 28th Ed. |
| | 1982, pg 638. |
| | Compiled by: |
| Helen Wong 3/91. Modified by Megan McGirr | , December 03 |

Sterilisation methods used: Sterile filtration with Millex FG 0.22 micron

Dry heat sterilisation 160° C. 1 hour

| | | |
|---|---|---|
| Ingredients | Unit Quantity | Manufacturer |

PART A

| | | |
|---|---|---|
| Retinoic acid injection (R-2625) | 100 mg | Sigma-Aldrich |
| Dehydrated alcohol 100% injection | 20 mL | DBL |

PART B

| | | |
|---|---|---|
| Castor oil | 800 mL | Sigma |

Method:

1. In 2×400 mL lots, sterilise castor oil in the dry heat steriliser at 160° C. for 1 hour, then allow to cool.

2. In the LAFH using a syringe dissolve 100 mg of Retinoic acid injection with 20 mL dehydrated alcohol. Shake well.

3. Attach a Millex FG filter to the syringe containing the Retinoic acid solution. Prime the Millex FG filter with solution then 8 mL to each 400 mL lot of castor oil. Stir vigorously with a stirring rod to ensure the solution is well dispersed throughout the castor oil.

4. Aseptically distribute 10 mL lots into each sterile 15 mL glass eye dropper bottle. Seal cap tightly

5. Seal bottle with tamper proof tape.

### Example 2

### Preparation of Interferon Formulation for Topical Application

Interferon Eye Drops 1 million units/mL (with Cresol)

Use:

Used for the treatment of Conjunctival and corneal neoplasm. Treatment duration is 6-8 weeks, depending upon response. Usual dose is 1 drop four times daily.

Requirements: 1×Interferon alpha 2b (Schering Plough) (Intron A) 18 million units redipen. 1×15 mL hypromellose eye drops 0.5% 1×15 ml sterile eye dropper bottle

Method of Preparation:

1. Remove cap of Intron® redipen and withdraw 0.67 mL (10 million units) from rubber bung (similar to Actrapid® penfill).

2. Dilute 10 mL with 0.5% Hypromellose eye drops. Mix well

3. Attach a 5 micron filter needle and filter contents of syringe into the sterile eye dropper bottle.

### Example 3

### Treatment of Oral Carcinoma

A patient who was formerly a smoker has had a squamous cell carcinoma previously excised from the floor of his mouth. A number of areas of dysplasia have been noted in the mouth and on the tongue and have the typical appearance of leukoplakia. Some of the lesions have been biopsied and although the pathology shows no frank malignancy, several areas show some atypia of the cells. Clinically, although the lesions look stable, they remain of concern.

Topical treatment is commenced. Lozenges containing interferon and retinoic acid are administered twice per day. The suspicious lesion is under the tongue and the patient is asked to hold the lozenge in this position using his tongue until it melts. The lesion begins to resolve after 6 weeks and by six months the appearance of the oral mucosa has normalized.

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

We claim:

1. A method for the treatment of a lesion originating in an epithelial layer in a subject, said lesion selected from a proliferative, pre-cancerous or cancerous lesion, the method comprising administering to the lesion of the subject, a therapeutically effective amount of an interferon α and a retinoid.

2. A method according to claim 1 wherein administration of the interferon α and the retinoid is sequential.

3. A method according to claim 2 wherein the time interval between interferon α administration and retinoid administration is up to 36 hours.

4. A method according to claim 3 wherein the time interval between interferon α administration and retinoid administration is from 24 to 36 hours.

5. A method according to claim 1 wherein administration of the interferon α and the retinoid is concurrent.

6. A method according to claim 1 wherein the interferon α and the retinoid are administered topically.

7. A method according to claim 1 wherein the interferon α is administered by injection into the lesion.

8. A method according to claim 1 wherein the interferon α is α-2b.

9. A method according to claim 1 wherein the retinoid is retinoic acid, all-trans retinoic acid, 9-cis retinoic acid or a compound of the formula:

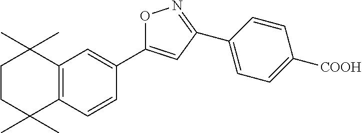 [SMILES: CC1(C)CCC(C)(C)c2cc(-c3cc(-c4ccc(C(=O)O)cc4)no3)ccc21]

10. A method according to claim 1 wherein the interferon α is administered in an amount from about 0.2 million to about 5 million international units or from about 1 million to about 3 million international units.

11. A method according to claim 1 wherein the retinoid is administered in an amount from about 0.01 to about 0.15% w/w or from about 0.05 to about 0.1% w/w.

12. A method according to claim 1 wherein said proliferative, pre-cancerous or cancerous lesion comprises a proliferative, pre-cancerous or cancerous lesion of the skin, bladder, a vaginal or cervical neoplasia or oral leukoplakia.

13. A topical formulation comprising or consisting essentially of interferon α and a retinoid for the combination treatment of a lesion originating in an epithelial layer selected from a proliferative, pre-cancerous or cancerous lesion in the epithelial layer, and a pharmaceutically acceptable carrier or excipient.

14. The topical formulation according to claim 13 wherein the interferon α is α-2b.

15. The topical formulation according to claim 13 wherein the retinoid is retinoic acid, all-trans retinoic acid, 9-cis retinoic acid, or a compound of the formula:

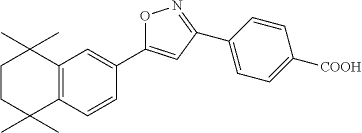 [SMILES: CC1(C)CCC(C)(C)c2cc(-c3cc(-c4ccc(C(=O)O)cc4)no3)ccc21]

16. The topical formulation according to claim 13 wherein said proliferative, pre-cancerous or cancerous lesion is a proliferative, pre-cancerous or cancerous lesion of the skin or bladder, a vaginal or cervical neoplasia, or an oral leukoplakia.

17. The topical formulation according to claim 13 wherein said proliferative, pre-cancerous or cancerous lesion comprises a proliferative, pre-cancerous or cancerous lesion of the skin, bladder, a vaginal or cervical neoplasia or oral leukoplakia.
